(19) United States
(12) Patent Application Publication
(10) Pub. No.: US 2018/0221599 A1
(43) Pub. Date: Aug. 9, 2018

(54) SURGICAL APPARATUS AND SMOKE DISCHARGING SYSTEM

(71) Applicant: OLYMPUS CORPORATION, Tokyo (JP)

(72) Inventors: Kunitoshi HIRAGA, Tama-shi (JP); Nobuyuki FURUKAWA, Hachioji-shi (JP); Satoshi HOMMA, Hino-shi (JP); Susumu AONO, Hachioji-shi (JP); Takanori USHIJIMA, Tama-shi (JP)

(73) Assignee: OLYMPUS CORPORATION, Tokyo (JP)

(21) Appl. No.: 15/945,981

(22) Filed: Apr. 5, 2018

Related U.S. Application Data
(63) Continuation of application No. PCT/JP2016/076462, filed on Sep. 8, 2016.

(30) Foreign Application Priority Data
Oct. 7, 2015 (JP) 2015-199519

(51) Int. Cl. A61M 13/00; A61B 18/14

(52) U.S. Cl. CPC A61M 13/006; A61B 2018/00595; A61B 18/14

(57) ABSTRACT

A surgical apparatus includes: a pipe sleeve section through which gas can circulate; a first seal section provided on a proximal end side of the pipe sleeve section; a second seal section provided on an insertion opening side of the pipe sleeve section; and a sheath provided with, between the first seal section and the second seal section, a hole portion through which gas circulates.

### CROSS REFERENCE TO RELATED APPLICATION

This application is a continuation application of PCT/JP2016/076462 filed on Sep. 8, 2016 and claims benefit of Japanese Application No. 2015-199519 filed in Japan on Oct. 7, 2015, the entire contents of which are incorporated herein by this reference.

### BACKGROUND OF THE INVENTION

### 1. Field of the invention

Embodiments of the present invention relate to a surgical apparatus and a smoke discharging system, and particularly relate to a surgical apparatus and a smoke discharging system each configured to feed and suck gas by using a sheath.

### 2. Description of the Related Art

Conventionally, endoscopes have been widely used in diagnosis and medical treatment of disease in a medical field. Such an endoscope is inserted into a body of a patient so that a surgeon can perform diagnosis and treatment while watching an image obtained through the endoscope. Recently, endoscopes have been used for medical treatment in an abdominal cavity in which a trocar is tapped.

For example, an endoscope is inserted into an abdominal cavity through one of two trocars tapped in a body wall of a patient, and a treatment instrument is inserted into the abdominal cavity of the patient through the other trocar. In this state, the surgeon operates the treatment instrument while watching an endoscope image and performs treatment of an affected part inside the abdominal cavity.

During an operation, a predetermined gas such as carbon dioxide is fed into the abdominal cavity of a patient by an air feeding apparatus, and observation and treatment of the affected part are performed in a space formed by the feeding. When medical treatment is performed in the abdominal cavity in such a state, use of an electrocautery scalpel, an ultrasound treatment instrument or the like generates smoke and mist in cauterization of the affected part, which blocks an operative field of an endoscope. In a developed circulation smoke discharging apparatus (refer to Japanese Patent Application Laid-Open Publication No. 11-318909, for example) configured to remove the generated smoke and mist through smoke discharging processing, the smoke and mist are removed from carbon dioxide sucked from inside of a body cavity by using a filter, and the carbon dioxide is fed into the body cavity again.

When smoke discharging processing is performed by the circulation smoke discharging apparatus disclosed in Japanese Patent Application Laid-Open Publication No. 11-318909, it is easy to control a balance between suction and air feeding and possible to prevent pulsing of the body cavity. Since carbon dioxide sucked from the inside of the body cavity is fed into the body cavity again, a use amount of carbon dioxide can be reduced.

In a recently developed method, a sheath is attached to an endoscope or a treatment instrument and inserted into a trocar together with the endoscope or the treatment instrument to perform a procedure. A distal end portion of the sheath is inserted further inside the body cavity than a distal end portion of the trocar, and thus close to a distal end of the endoscope or treatment instrument. Thus, smoke can be more efficiently discharged by sucking carbon dioxide inside the body cavity and feeding carbon dioxide into the body cavity through the distal end of the sheath, not through the distal end of the trocar, than a case in which the suction and feeding are performed through the trocar.

### SUMMARY OF THE INVENTION

A surgical apparatus according to an aspect of the present invention includes: a pipe sleeve section through which gas can circulate; a first seal section provided on a proximal end side of the pipe sleeve section; a second seal section provided on an insertion opening side of the pipe sleeve section; and a sheath provided with, between the first seal section and the second seal section, a hole portion through which gas circulates.

A surgical apparatus according to another aspect of the present invention includes: a first trocar provided with a check valve on a proximal end side and connected with an air feeding tube; a second trocar provided with a check valve on a proximal end side and connected with a suction tube; and a sheath provided with a ring-shaped seal member on an outer peripheral surface on a distal end side. The sheath is inserted into at least one of the first trocar and the second trocar. The seal member holds airtightness between the sheath and the first trocar and/or the second trocar in which the sheath is inserted. A hole portion through which gas circulates is provided between the check valve and the seal member.

A smoke discharging system according to one aspect of the present invention includes: a first trocar that is connected with one end of an air feeding tube having another end connected with an air feeding apparatus and is provided with a check valve on a proximal end side; a second trocar that is connected with one end of a suction tube having another end connected with a suction apparatus and is provided with a check valve on a proximal end side; and a sheath provided with a ring-shaped seal member on an outer peripheral surface on a distal end side. The sheath is inserted into at least one of the first trocar and the second trocar. The seal member holds airtightness between the sheath and the first trocar and/or the second trocar in which the sheath is inserted. A hole portion through which gas circulates is provided between the check valve and the seal member.

### BRIEF DESCRIPTION OF THE DRAWINGS

FIG. 1 is a diagram for description of an exemplary entire configuration of a surgical system including a smoke discharging system according to a first embodiment of the present invention;

FIG. 2 is a schematic diagram for description of an exemplary configuration of a sheath 13;

FIG. 3 is a diagram for description of a state in which the sheath 13 is inserted in a trocar 12c;

FIG. 4 is a diagram for description of flow of carbon dioxide at suction while the sheath 13 is inserted;

FIG. 5 is a diagram for description of the flow of carbon dioxide at suction while the sheath 13 is removed;

FIG. 6 is a diagram for description of another exemplary entire configuration of the surgical system including the smoke discharging system according to the first embodiment of the present invention;

FIG. 7 is a diagram for description of the flow of carbon dioxide at air feeding while the sheath 13 is inserted;

FIG. 8 is a diagram for description of the flow of carbon dioxide at air feeding while the sheath 13 is removed;

FIG. 9 is a diagram for description of yet another exemplary entire configuration of the surgical system including the smoke discharging system according to the first embodiment of the present invention;

FIG. 10 is a schematic diagram for description of an exemplary configuration of a sheath 13a according to a second embodiment of the present invention;

FIG. 11 is a schematic diagram for description of another exemplary configuration of a sheath 13b according to the second embodiment of the present invention;

FIG. 12 is a schematic diagram for description of yet another exemplary configuration of a sheath 13c according to the second embodiment of the present invention;

FIG. 13 is a schematic diagram for description of yet another exemplary configuration of a sheath 13d according to the second embodiment of the present invention;

FIG. 14 is a schematic diagram for description of an exemplary configuration of a sheath 13′ according to a third embodiment of the present invention;

FIG. 15 is a diagram for description of the flow of carbon dioxide at suction while a treatment instrument 11 is inserted; and

FIG. 16 is a diagram for description of the flow of carbon dioxide at suction while the treatment instrument 11 is removed.

### DETAILED DESCRIPTION OF THE PREFERRED EMBODIMENTS

Embodiments will be described below with reference to accompanying drawings.

### First Embodiment

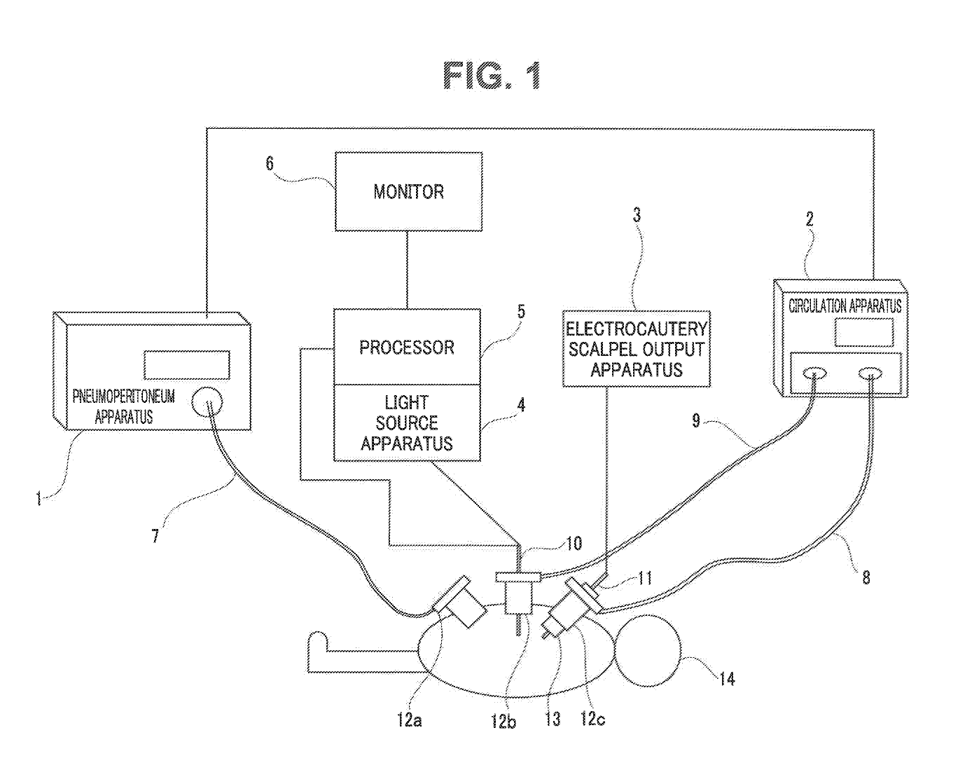

FIG. 1 is a diagram for description of an exemplary entire configuration of a surgical system including a smoke discharging system according to a first embodiment of the present invention. As illustrated in FIG. 1, the surgical system according to the present embodiment is used in an operation in which an affected part inside the abdominal cavity of a patient, which is expanded by feeding, for example, carbon dioxide in endoscope observation is treated by using a treatment instrument such as an electrocautery scalpel 11.

As illustrated in FIG. 1, an air feeding trocar 12a, an endoscope insertion trocar 12b as a first trocar, and a treatment instrument insertion trocar 12c as a second trocar are tapped in an abdominal wall of a patient 14. A sheath 13 is inserted in the abdominal cavity through the trocar 12c, and an endoscope 10 is inserted in the abdominal cavity through the trocar 12b. The electrocautery scalpel 11 is disposed through the sheath 13.

The sheath 13 arid the electrocautery scalpel 11 are mechanically integrated with each other. With this configuration, the sheath 13 is constantly disposed near a distal end of the electrocautery scalpel 11 when the electrocautery scalpel 11 is moved forward and backward relative to a distal end portion of the trocar 12b to cauterize an affected part with the electrocautery scalpel 11, which enables efficient smoke discharging.

The endoscope 10 is connected with a light source apparatus 4 and a processor 5. The light source apparatus 4 supplies illumination light to the endoscope 10 as light emitted from a semiconductor light source is guided through a light guiding member, and for example, the color and light intensity distribution of the light are converted through an optical conversion member provided at a distal end of the light guiding member. The processor 5 supplies power voltage to the endoscope 10, and processes an image picked up by the endoscope 10 and causes a monitor 6 to display the image.

The electrocautery scalpel 11 is connected with an electrocautery scalpel output apparatus 3, The electrocautery scalpel output apparatus 3 outputs high-frequency current that generates high-frequency electric energy. When an electrode at a distal end of the electrocautery scalpel 11 is made contact with the tissue of an affected part of the patient 14, the high-frequency current outputted from the electrocautery scalpel output apparatus 3 intensively flows through the tissue of the affected part to generate Joule heat. The heat is used to perform, for example, dissection of the tissue of the affected part or hemostasis coagulation of a bleeding site.

A pneumoperitoneum apparatus 1 configured to feed a predetermined gas is connected with a tank (not illustrated) filled with carbon dioxide (CO₂ gas). The pneumoperitoneum apparatus 1 is connected with one end of a pneumoperitoneum tube 7. The other end of the pneumoperitoneum tube 7 is connected with the trocar 12a tapped in the abdominal wall of the patient 14. That is, the pneumoperitoneum apparatus 1 is configured to feed carbon dioxide into the abdominal cavity of the patient 14 through the pneumoperitoneum tube 7 and the trocar 12a.

A circulation apparatus 2 is connected with one end of a suction tube 8. The other end of the suction tube 8 is connected with the trocar 12c. The circulation apparatus 2 is connected with one end of an air feeding tube 9. The other end of the air feeding tube 9 is connected with the trocar 12b. The circulation apparatus 2 is provided with a filter (not illustrated) for removing smoke and mist. The circulation apparatus 2 sucks carbon dioxide filling the abdominal cavity of the patient 14 through the suction tube 8, the trocar 12c, and the sheath 13. Then, after the smoke and mist are removed from the carbon dioxide through the filter (not illustrated) in the circulation apparatus 2, the carbon dioxide is blown out near the endoscope 10 through the air feeding tube 9 and the trocar 12b.

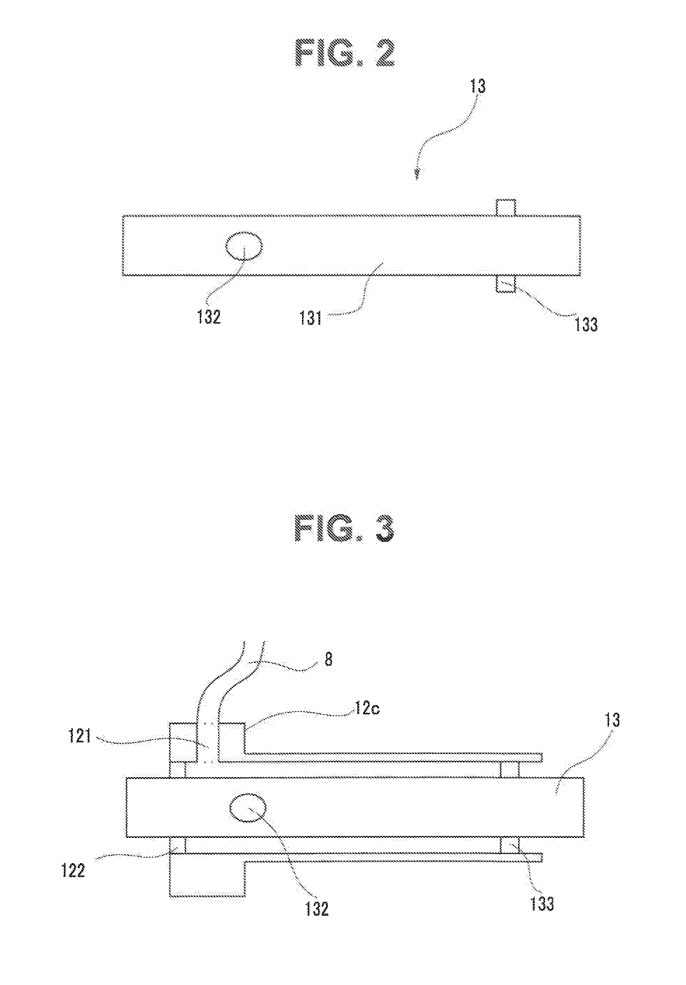

FIG. 2 is a schematic diagram for description of an exemplary configuration of the sheath 13. The sheath 13 includes a tubular sheath body 131 formed of a rigid resin pipe or the like through which a treatment instrument such as the electrocautery scalpel 11 can be inserted. A ring-shaped seal member 133 is attached to an outer peripheral surface of the sheath body 131 on a distal end side. The seal member 133 is an elastic member such as a silicon rubber member or an elastic O-shaped ring. A hole 132 through which gas passes is opened at an outer peripheral section of the sheath body 131 on a proximal end side. Note that the distance between the hole 132 and the seal member 133 is shorter than the length of the trocar 12c in the longitudinal direction. The sheath 13 includes a mechanism (not illustrated) for mechanical integration with the electrocautery scalpel 11. With this configuration, the sheath 13 is constantly disposed near the distal end of the electrocautery scalpel 11 even when the electrocautery scalpel 11 is moved forward arid backward relative to a distal end portion of the trocar 12c to cauterize an affected part with the electrocautery scalpel 11.

FIG. 3 is a diagram for description of a state in which the sheath 13 is inserted in the trocar 12c. A tube connection section 121 through which gas inside the trocar 12c passes is opened at an outer peripheral section of the trocar 12c as a hollow tubular member on a proximal end side. The tube connection section 121 is connected with the suction tube 8. A trocar check valve 122 is provided on a proximal end side of the tube connection section 121. The trocar check valve 122 seals a gap between an outer peripheral section of the sheath 13 and an inner peripheral section of the trocar 12c when the sheath 13 is disposed through a hollow region inside the trocar 12c, thereby preventing gas inside the trocar 12c from being sent out into air from the proximal end side, and air from flowing into inside the trocar 12c.

When the sheath 13 is disposed through the trocar 12c, the seal member 133 provided on an outer peripheral surface of the sheath 13 closely contacts with the inner peripheral section of the trocar 12c so that the gap between the outer peripheral section of the sheath 13 and the inner peripheral section of the trocar 12c is sealed. While the sheath 13 is disposed through the trocar 12c, the hole 132 is positioned between the trocar check valve 122 and the seal member 133.

Note that, the trocar 12c is not limited to the above-described configuration but may be any general-purpose trocar provided with the tube connection section 121 and the trocar check valve 122. For example, a trocar having a double structure in which a hollow pipe is provided in a coat pipe may be used.

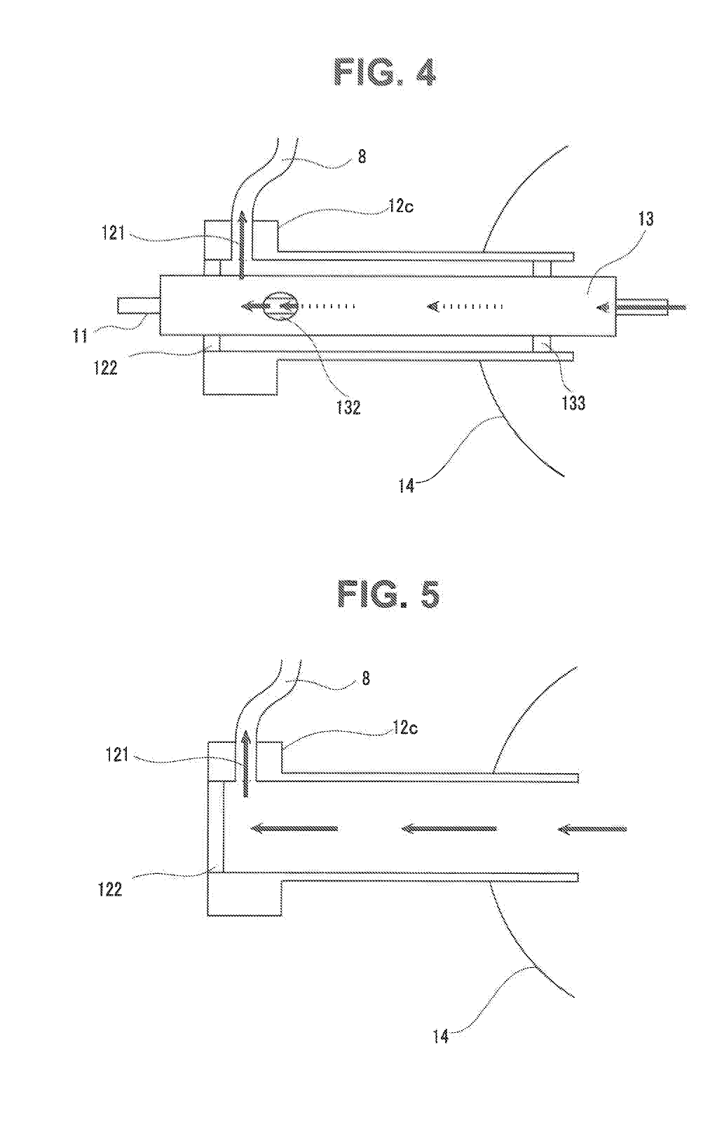

FIG. 4 is a diagram for description of a flow of carbon dioxide at suction while the sheath 13 is inserted. The trocar 12c is tapped in a body cavity of the patient 14, and the sheath 13 through which the electrocautery scalpel 11 is disposed is inserted in the trocar 12c. When the circulation apparatus 2 is driven, carbon dioxide inside the body cavity is sucked toward a proximal end side through an opening portion of the sheath 13 on a distal end side, passes through the hole 132, and is sent out into a space between the outer peripheral section of the sheath 13 and the inner peripheral section of the trocar 12c.

Note that, since the gap between the outer peripheral section of the sheath 13 and the inner peripheral section of the trocar 12c on a distal end side of the trocar 12c is sealed by the seal member 133, carbon dioxide inside the body cavity is not sucked through an opening portion of the trocar 12c on the distal end side but is sucked through the opening portion of the sheath 13 on the distal end side. That is, carbon dioxide can be sucked from near the electrocautery scalpel 11, which generates smoke, and thus smoke is efficiently discharged as compared to suction through the opening portion of the trocar 12c on the distal end side.

Since the gap between the outer peripheral section of the sheath 13 and the inner peripheral section of the trocar 12c on the proximal end side of the trocar 12c is sealed by the trocar check valve 122, air does not flow in and carbon dioxide is not discharged into air through the space between the outer peripheral section of the sheath 13 and the inner peripheral section of the trocar 12c. Thus, carbon dioxide inside the trocar 12c is sucked from the suction tube 8 through the tube connection section 121 without leaking from the proximal end side, and is sent to the circulation apparatus 2.

FIG. 5 is a diagram for description of the flow of carbon dioxide at suction while the sheath 13 is removed. For example, when the electrocautery scalpel 11 is cleaned during a procedure, the sheath 13 is removed from the trocar 12c while the electrocautery scalpel 11 is disposed through the sheath 13. As illustrated in FIG. 5, when the sheath 13 is removed from the trocar 12c, the seal member 133 is removed together with the sheath 13, and accordingly, the opening portion of the trocar 12c on the distal end side is left opened. Thus, carbon dioxide inside the body cavity can be sucked from the distal end side of the trocar 12c.

While the sheath 13 is removed, the trocar check valve 122 completely seals an opening portion of the trocar 12e at a proximal end portion to prevent gas inside the trocar 12c from being sent out from the proximal end side into air, and air from flowing into the trocar 12c. Thus, carbon dioxide inside the body cavity is sucked into the trocar 12c through the opening portion of the trocar 12c on the distal end side and sent out to the circulation apparatus 2 through the tube connection section 121 and the suction tube 8. That is, a suction pipe line between inside of the body cavity and the suction tube 8 remains in an airtight state against atmosphere while the sheath 13 is removed, thereby preventing inflow of air and outflow of carbon dioxide into air.

As described above, according to the present embodiment, since the sheath 13 is provided with the ring-shaped seal member 133 on the outer peripheral surface on the distal end side and provided with the hole 132, through which gas passes, opened at the outer peripheral section on the proximal end side, carbon dioxide inside the body cavity can be sucked through the opening portion of the sheath 13 on the distal end side while the sheath 13 is inserted in the trocar 12c, thereby improving smoke discharging efficiency. Since the suction tube 8 is connected with the tube connection section 121 of the trocar 12c, carbon dioxide inside the body cavity can be sucked through the opening portion of the trocar 12c on the distal end side while the sheath 13 is removed. The trocar check valve 122 can maintain airtightness of the opening portion of the trocar 12c on the proximal end side against atmosphere while the sheath 13 is inserted or removed, and thus smoke can be discharged without variation of abdominal cavity pressure.

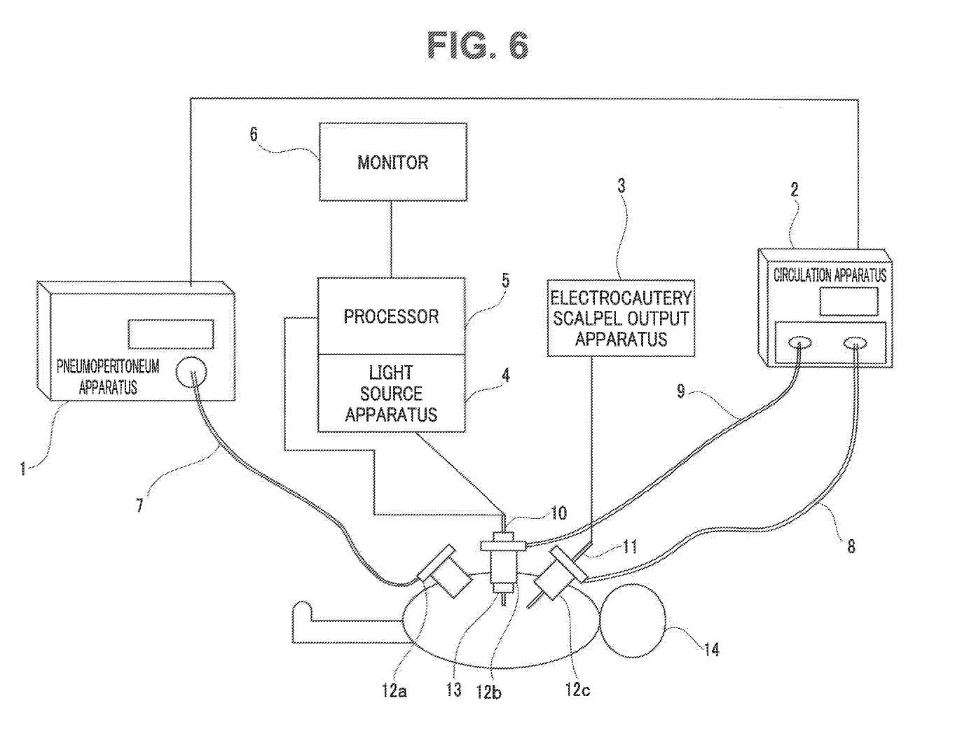

Note that the sheath 13 is inserted in the trocar 12c connected with the suction tube 8 in the above description but may be inserted in the trocar 12b connected with the air feeding tube 9. FIG. 6 is a diagram for description of another exemplary entire configuration of the surgical system including the smoke discharging system according to the first embodiment of the present invention. The surgical system illustrated in FIG. 6 is different from the surgical system illustrated in FIG. 1 in that the sheath 13 is inserted in the trocar 12b, not in the trocar 12c.

In the surgical system illustrated in FIG. 6, carbon dioxide including smoke generated by the electrocautery scalpel 11 is sucked through the opening portion of the trocar 12c on the distal end side. The carbon dioxide having been subjected to smoke discharging by the circulation apparatus 2 is fed from the opening portion of the sheath 13 on the distal end side through the air feeding tube 9, the trocar 12b, and the sheath 13. Note that the sheath 13 has a configuration identical to the configuration of the sheath 13 illustrated in FIG. 2. The trocar 12b has a configuration identical to the configuration of the trocar 12c described with reference to FIG. 3 except that the tube connection section 121 is connected with the air feeding tube 9, not with the suction tube 8.

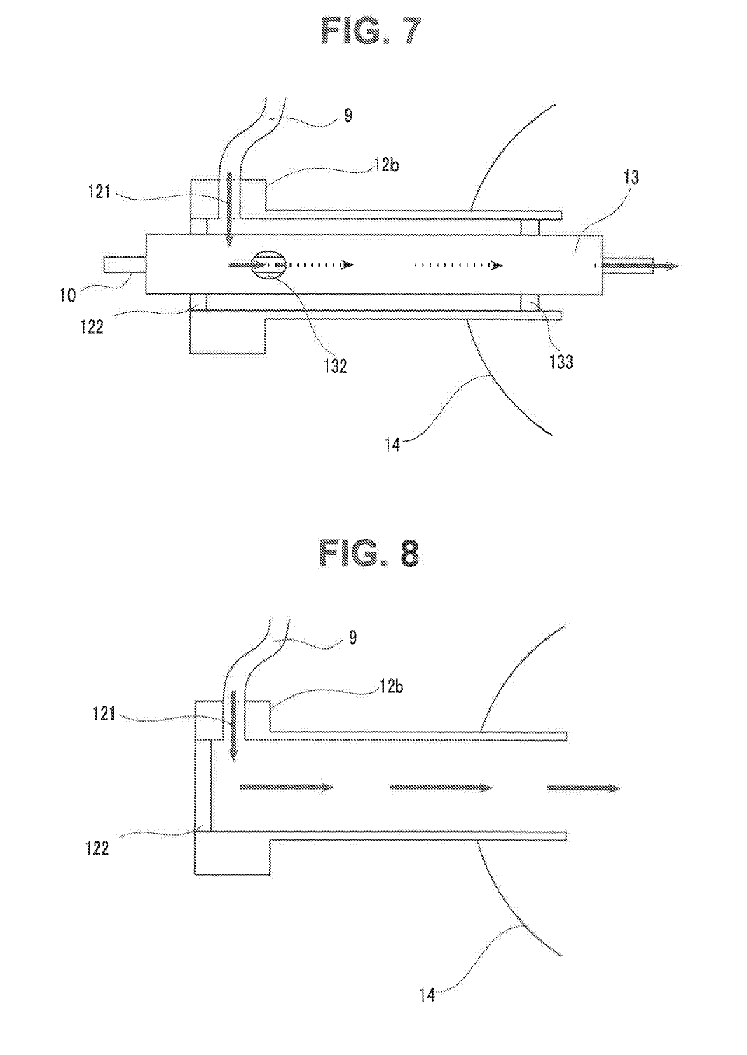

FIG. 7 is a diagram for description of the flow of carbon dioxide at air feeding while the sheath 13 is inserted. The trocar 12b is tapped in the body cavity of the patient 14, and the sheath 13 through which the endoscope 10 is disposed is inserted in the trocar 12b. The sheath 13 includes a mechanism (not illustrated) for mechanical integration with the endoscope 10. With this configuration, the sheath 13 is constantly disposed near the distal end of the endoscope 10 even when the endoscope 10 is moved forward and backward relative to the distal end portion of the trocar 12b. When the circulation apparatus 2 is driven, carbon dioxide inside the body cavity is subjected to smoke discharging at the circulation apparatus 2, and then sent out into a space between the outer peripheral section of the sheath 13 and an inner peripheral section of the trocar 12b from the tube connection section 121 through the air feeding tube 9. Then, the carbon dioxide having been subjected to the smoke discharging passes through the hole 132 and an internal space of the sheath 13, and is sprayed toward the inside of the body cavity from the opening portion of the sheath 13 on the distal end side.

In this state, a gap between the outer peripheral section of the sheath 13 and the inner peripheral section of the trocar 12b on a distal end side of the trocar 12b is sealed by the seal member 133, and thus, the carbon dioxide fed from the circulation apparatus 2 is not sprayed from an opening portion of the trocar 12b on the distal end side but is sprayed from the opening portion of the sheath 13 on the distal end side. That is, the carbon dioxide can be sprayed near a distal end portion of the endoscope 10, from which smoke is desired to be removed, and thus smoke is efficiently discharged as compared to spraying from the opening portion of the trocar 12b on the distal end side.

Since the gap between the outer peripheral section of the sheath 13 and the inner peripheral section of the trocar 12b on a proximal end side of the trocar 12b is sealed by the trocar check valve 122, air does not flow in and carbon dioxide is not discharged into air through the space between the outer peripheral section of the sheath 13 and the inner peripheral section of the trocar 12b. Thus, carbon dioxide inside the trocar 12b is sprayed into the body cavity through the hole 132 provided to the sheath 13 and the internal space of the sheath 13 without leaking from the proximal end side.

FIG. 8 is a diagram for description of the flow of carbon dioxide at air feeding while the sheath 13 is removed. For example, when the endoscope 10 is cleaned during a procedure, the sheath 13 is removed from the trocar 12b while the endoscope 10 is disposed through the sheath 13. As illustrated in FIG. 8, when the sheath 13 is removed from the trocar 12b, the seal member 133 is removed together with the sheath 13, and accordingly, the opening portion of the trocar 12b on the distal end side is opened. Thus, carbon dioxide can be sent into the body cavity from the distal end side of the trocar 12c.

While the sheath 13 is removed, the trocar check valve 122 completely seals an opening portion of the trocar 12b at a proximal end portion to prevent gas inside the trocar 12b from being sent from the proximal end side into air, and air from flowing into the trocar 12b. Thus, carbon dioxide fed from the air feeding tube 9 is sent into the body cavity from the opening portion of the trocar 12b on the distal end side through the tube connection section 121. That is, an air feeding pipe line between the air feeding tube 9 and the inside of the body cavity remains in an airtight state against atmosphere while the sheath 13 is removed, thereby preventing inflow of air and outflow of carbon dioxide into air.

As described above, since the sheath 13 provided with the ring-shaped seal member 133 on the outer peripheral surface on the distal end side and provided with the hole 132, through which gas passes, opened at the outer peripheral section on the proximal end side, is used in the trocar 12b side in which the endoscope 10 is inserted, carbon dioxide can be blown out near the distal end of the endoscope 10 from the opening portion of the sheath 13 on the distal end side, thereby improving the smoke discharging efficiency. Since the air feeding tube 9 is connected with the tube connection section 121 of the trocar 12b, carbon dioxide can be sent into the body cavity through the opening portion of the trocar 12b on the distal end side while the sheath 13 is removed. The trocar check valve 122 can maintain airtightness of the opening portion of the trocar 12b on the proximal end side against atmosphere while the sheath 13 is inserted or removed, and thus smoke can be discharged without variation of the abdominal cavity pressure.

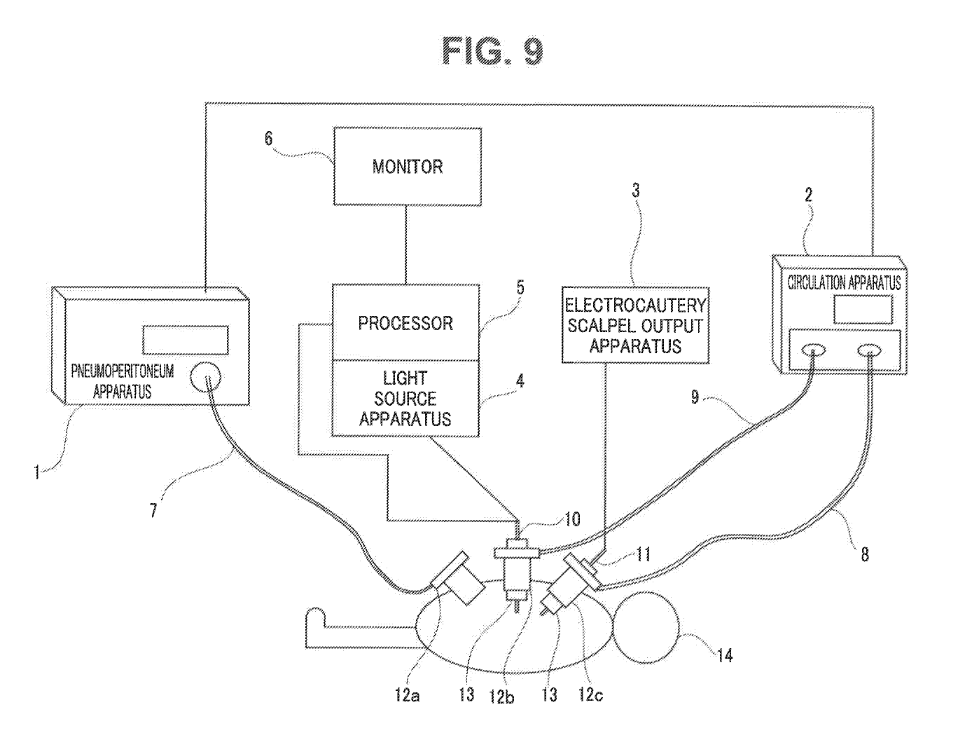

Note that the sheath 13 illustrated in FIG. 2 may be inserted in each of the trocar 12b and the trocar 12c. FIG. 9 is a diagram for description of another exemplary entire configuration of the surgical system including the smoke discharging system according to the first embodiment of the present invention. As illustrated in FIG. 9, when the sheath 13 is used in each of the trocar 12b, in which the endoscope 10 is inserted, and the trocar 12c, in which the electrocautery scalpel 11 is inserted, carbon dioxide can be sucked near the electrocautery scalpel 11, which generates smoke, subjected to smoke discharging processing by the circulation apparatus 2, and sprayed near the distal end of the endoscope 10, thereby improving the smoke discharging efficiency.

### Second Embodiment

In the above-described sheath 13 according to the first embodiment, the ring-shaped seal member 133 provided on the outer peripheral surface on the distal end side is, for example, an O-shaped ring having a structure in such a shape that a contact area of the outer peripheral surface of the sheath 13 is equal to a contact area of each trocar 12. However, the seal member 133 has a different shape in the present embodiment. Note that the entire configuration of the surgical system including the sheath 13 is same as the entire configuration in the first embodiment.

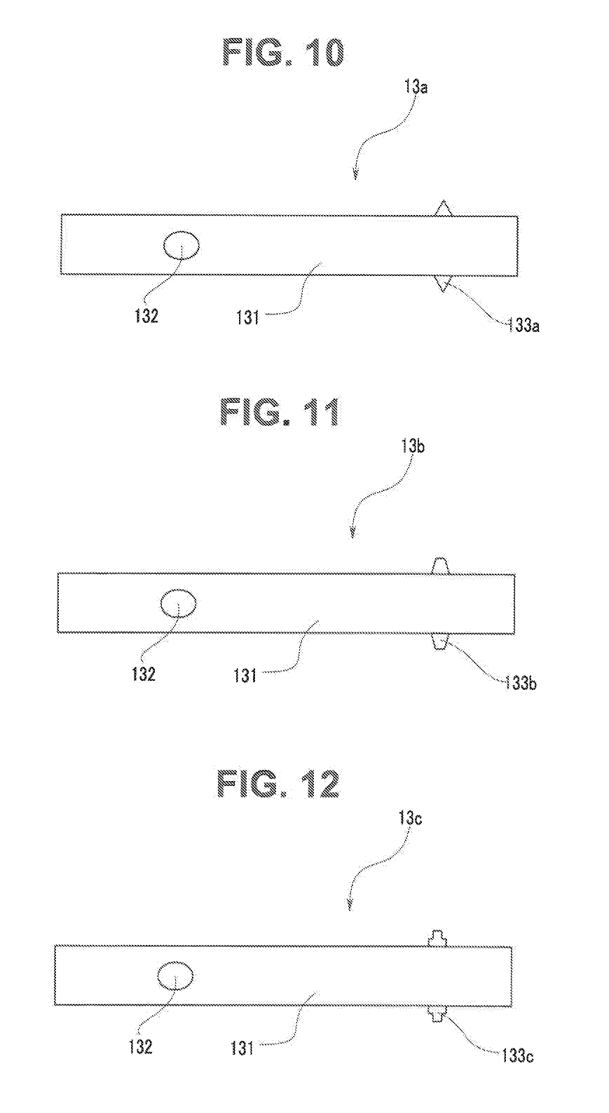

FIG. 10 is a schematic diagram for description of an exemplary configuration of a sheath 13a according to a second embodiment of the present invention. As illustrated in FIG. 10, in a ring-shaped seal member 133a attached to the sheath 13a, a section parallel to a longitudinal direction of the sheath 13a is shaped in a triangle having a base at an outer peripheral surface of the sheath 13a. In this manner, the seal member 133a is formed so that the section parallel to the longitudinal direction of the sheath 13a has a smaller area at a position farther away from the outer peripheral surface of the sheath 13a in a circumferential direction, and thus the seal member 133a has a small contact area with the trocar 12. This configuration leads to reduction of friction due to the seal member 133a when the sheath 13a is inserted into or removed from the trocar 12, which facilitates insertion and removal and improves operability.

Note that the shape of the seal member 133a is not limited to the shape illustrated in FIG. 10 but may have any other shape that leads to reduction of contact area between the seal member 133a and the trocar 12. FIGS. 11 and 12 are each a schematic diagram for description of another exemplary configuration of the sheath according to the second embodiment of the present invention. For example, a seal member 133b may have a trapezoid section parallel to the longitudinal direction of the sheath 13a as illustrated in FIG. 11. For example, a seal member 133c may have a convex shape provided with a stepped part at a halfway position as illustrated in FIG. 12.

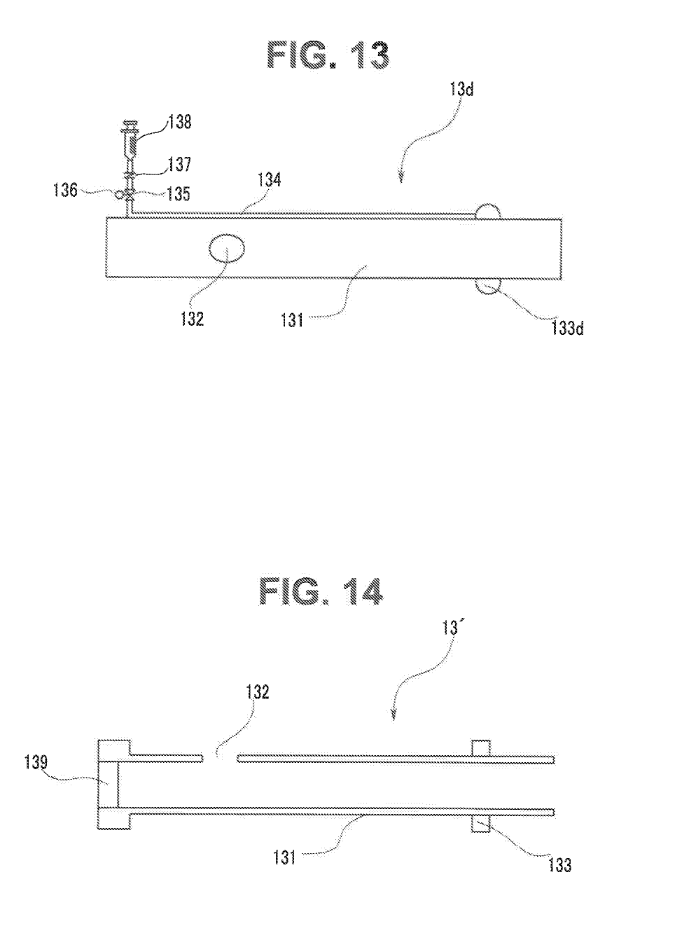

Alternatively, the shape of the seal member 133 does not need to be fixed but may be changed, or expanded and contracted in accordance with a diameter and a friction degree of the trocar 12, which is inserted and removed. FIG. 13 is a schematic diagram for description of another exemplary configuration of a sheath 13d according to the second embodiment of the present invention. As illustrated in FIG. 13, a ring-shaped seal member 133d attached to an outer peripheral surface of the sheath 13d near a distal end portion is an elastic deformable member made of, for example, silicon. The seal member 133d has a hollow structure and is connected with one end of a pipe line 134 provided on the outer peripheral surface of the sheath 13d along a longitudinal direction. The other end of the pipe line 134 is connected with a cylinder 138 through a relief valve 135 and a check valve 137.

That is, the sheath 13d illustrated in FIG. 13 has a configuration that allows gas to be injected from the cylinder 138 into the seal member 133d. To reduce a size of the seal member 133d, a relief button 136 provided to the relief valve 135 is pressed to relieve the gas injected in the seal member 133d. In this manner, the size of the seal member 133d can be changed by adjusting an amount of the injected gas, which eliminates need to prepare a plurality of sheaths 13 in accordance with different diameters of the trocar 12, and thus leads to cost reduction.

### Third Embodiment

In the above-described first embodiment, the sheath 13 is removed when the electrocautery scalpel 11 or the endoscope 10 is removed from the trocar 12b or 12c. However, the present embodiment is different from the first embodiment in that only the electrocautery scalpel 11 or the endoscope 10 is removed while the sheath 13 is mounted on the trocars 12b and 12c. Note that the entire configuration of the surgical system including the sheath 13 is same as the entire configuration in the first embodiment.

FIG. 14 is a schematic diagram for description of an exemplary configuration of a sheath 13′ according to a third embodiment of the present invention. The sheath 13′ includes a sheath check valve 139 provided to an opening portion at a proximal end portion in addition to the hole 132 provided to a side surface section and the seal member 133. The sheath check valve 139 seals a gap between an inner peripheral section of the sheath 13′ and the outer peripheral section of the electrocautery scalpel 11 or the endoscope 10 when the electrocautery scalpel 11 or the endoscope 10 is disposed through a hollow region inside the sheath 13′, thereby preventing gas inside the sheath 13′ from being sent into air from a proximal end side, and air from flowing into the sheath 13′. The sheath check valve 139 entirely covers an opening portion of the sheath 13′ on the proximal end side when the electrocautery scalpel 11 or the endoscope 10 is removed from the sheath 13′, and holds airtightness to prevent inflow of air and outflow of carbon dioxide.

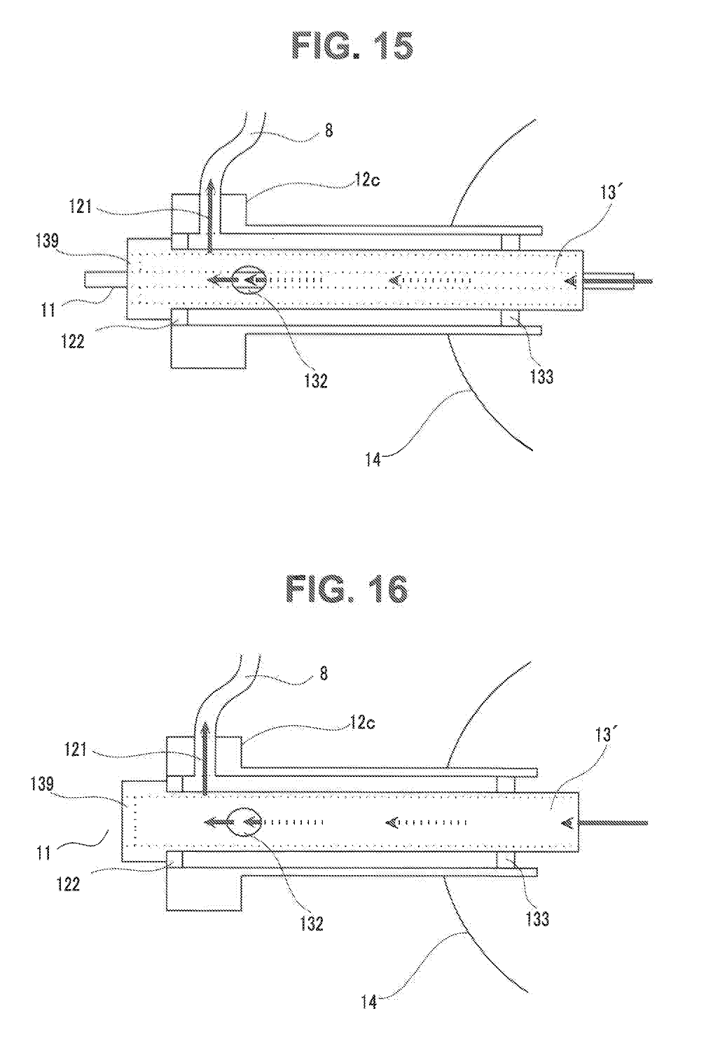

FIG. 15 is a diagram for description of the flow of carbon dioxide at suction while the sheath 13′ is inserted. The trocar 12c is tapped in the body cavity of the patient 14, and the sheath 13′ through which the electrocautery scalpel 11 is disposed is inserted in the trocar 12c. When the circulation apparatus 2 is driven, carbon dioxide inside the body cavity is sucked toward the proximal end side through an opening portion of the sheath 13′ on a distal end side and sent out to a space between an outer peripheral section of the sheath 13′ and the inner peripheral section of the trocar 12c through the hole 132.

Note that, since a gap between the outer peripheral section of the sheath 13′ and the inner peripheral section of the trocar 12c on the distal end side of the trocar 12c is sealed by the seal member 133, carbon dioxide inside the body cavity is not sucked through the opening portion of the trocar 12c on the distal end side but is sucked through the opening portion of the sheath 13′ on the distal end side. That is, carbon dioxide can be sucked near the electrocautery scalpel 11, which generates smoke, and thus smoke is efficiently discharged as compared to suction through the opening portion of the trocar 12c on the distal end side.

Since the gap between the outer peripheral section of the sheath 13′ and the inner peripheral section of the trocar 12c on the proximal end side of the trocar 12c is sealed by the trocar check valve 122, air does not flow in and carbon dioxide is not discharged into air through the space between the outer peripheral section of the sheath 13′ and the inner peripheral section of the trocar 12c. Since the gap between the inner peripheral section of the sheath 13′ and the outer peripheral section of the electrocautery scalpel 11 is sealed by the sheath check valve 139, air does not flow in and carbon dioxide is not discharged into air through the opening portion of the sheath 13′ on the proximal end side. Thus, carbon dioxide inside the trocar 12c is sucked from the suction tube 8 through the tube connection section 121 without leaking from the proximal end side, and is sent to the circulation apparatus 2.

FIG. 16 is a diagram for description of the flow of carbon dioxide at suction while the electrocautery scalpel 11 is removed. In the present embodiment, the sheath 13′ remains inserted in the trocar 12c when the electrocautery scalpel 11 is removed from the trocar 12c. While the electrocautery scalpel 11 is removed, the sheath check valve 139 completely seals the opening portion of the sheath 13′ on the proximal end side to prevent inflow of air and outflow of gas inside the sheath 13′ and the trocar 12c into air from the proximal end side of the sheath 13′.

Thus, carbon dioxide inside the body cavity is sucked from the opening portion of the sheath 13′ on the distal end side into the trocar 12c through the hole 132 and sent out to the circulation apparatus 2 through the tube connection section 121 and the suction tube 8. That is, the suction pipe line between the inside of the body cavity and the suction tube 8 remains in an airtight state against atmosphere while the electrocautery scalpel 11 is removed, thereby preventing inflow of air and outflow of carbon dioxide into air.

In this manner, according to the present embodiment, the ring-shaped sheath check valve 139 is provided at the opening portion of the sheath 13′ on the proximal end side so that the opening portion of the sheath 13′ on the proximal end side is sealed to maintain airtightness against atmosphere while the electrocautery scalpel 11 is removed, and thus smoke can be discharged without variation of the abdominal cavity pressure. Since the sheath 13′ remains inserted in the trocar 12c while the electrocautery scalpel 11 is removed, carbon dioxide can be sucked through an opening portion of the sheath 13′ at a distal end, which leads to further improvement of the smoke discharging efficiency.

Note that the sheath 13′ according to the present embodiment may be used for the trocar 12b into which the endoscope 10 is inserted. Alternatively, the sheath 13′ may be used for both of the trocars 12b and 12c.

Each “section” in the present specification conceptually corresponds to a function of the embodiments, and does not necessarily directly correspond to a particular hardware piece or software routine. Thus, in the present specification, the embodiments are described on assumption of virtual circuit blocks (sections) having the corresponding functions of the embodiments. Steps in each procedure in the present embodiment may be executed in any different order, may be simultaneously executed, or may be executed in different orders between executions as long as features of the procedure are maintained. All or part of the steps of each procedure in the present embodiment may be achieved by hardware.

Although some embodiments of the present invention are described above, these embodiments are merely exemplary and not intended to limit the scope of the invention. These novel embodiments may be implemented in various other forms involving various kinds of omissions, replacements, and changes without departing the spirit of the invention. These embodiments and modifications are included in the scope and spirit of the invention and also included in the invention recited in the claims and equivalents of the invention.

What is claimed is:

1. A surgical apparatus comprising:
a pipe sleeve section through which gas can circulate;
a first seal section provided on a proximal end side of the pipe sleeve section;
a second seal section provided on an insertion opening side of the pipe sleeve section; and
a sheath provided with, between the first seal section and the second seal section, a hole portion through which gas circulates.

2. A surgical apparatus comprising:
a first trocar provided with a check valve on a proximal end side and connected with an air feeding tube;
a second trocar provided with a check valve on a proximal end side and connected with a suction tube; and
a sheath provided with a ring-shaped seal member on an outer peripheral surface on a distal end side and integrated with a treatment instrument,
wherein
the sheath is inserted into at least one of the first trocar and the second trocar,
the seal member holds airtightness between the sheath and the first trocar and/or the second trocar in which the sheath is inserted, and
a hole portion through which gas circulates is provided between the check valve and the seal member.

3. The surgical apparatus according to claim 1, further comprising:
a first trocar provided with a check valve on a proximal end side and connected with an air feeding tube; and
a second trocar provided with a check valve on a proximal end side and connected with a suction tube,
wherein
the sheath is provided with, on a proximal end side, the hole portion through which gas circulates and the ring-shaped second seal section on an outer peripheral surface on a distal end side and is integrated with a treatment instrument, and
the sheath is inserted into at least one of the first trocar and the second trocar, and
the second seal section holds airtightness between the sheath and the first trocar and/or the second trocar in which the sheath is inserted.

4. The surgical apparatus according to claim 1, wherein the second seal section has a cross-sectional area decreasing in a direction from the sheath toward the trocar.

5. The surgical apparatus according to claim 1, wherein the second seal section has a variable size.

6. The surgical apparatus according to claim 5, wherein the sheath includes a gas injection section configured to inject gas into the second seal section, and a relief section configured to discharge gas from the second seal section.

7. A smoke discharging system comprising:
a first trocar that is connected with one end of an air feeding tube having another end connected with an air feeding apparatus and is provided with a check valve on a proximal end side;
a second trocar that is connected with one end of a suction tube having another end connected with a suction apparatus and is provided with a check valve on a proximal end side; and
a sheath provided with a ring-shaped seal member on an outer peripheral surface on a distal end side,
wherein
the sheath is inserted into at least one of the first trocar and the second trocar,
the seal member holds airtightness between the sheath and the first trocar and/or the second trocar in which the sheath is inserted, and
a hole portion through which gas circulates is provided between the check valve and the seal member.

8. The smoke discharging system according to claim 7, wherein the air feeding apparatus and the suction apparatus are configured as a circulation smoke discharging apparatus configured to perform circulation smoke discharging by feeding a predetermined gas and sucking smoke generated in a subject and gas inside the subject.

9. The smoke discharging system according to claim 7, wherein the gas is sucked and/or fed through an opening portion of the first trocar and/or the second trocar on a distal end side when the sheath is removed from the first trocar and/or the second trocar.

10. The smoke discharging system according to claim 7, wherein the seal member has a cross-sectional area decreasing in a direction from the sheath toward the trocar.

11. The smoke discharging system according to claim 7, wherein the sheath is provided with a check valve at an opening portion on a proximal end side.

12. The smoke discharging system according to claim 7, wherein the seal member has a variable size.

13. The smoke discharging system according to claim 12, wherein the sheath includes a gas injection section configured to inject gas into the seal member, and a relief section configured to discharge gas from the seal member.
